(19) United States
(12) Patent Application Publication
(10) Pub. No.: US 2026/0285795 A1
(43) Pub. Date: Sep. 24, 2026

(54) LIQUID PHASE PROCESS FOR PREPARING ISOPHORONE

(71) Applicant: Evonik Operations GmbH, Essen (DE)

(72) Inventors: Jan Benedikt Metternich, Recklinghausen (DE); Jörg-Joachim Nitz, Essen (DE); Melanie Wieser, Gelsenkirchen (DE); Maximilian Hanasek, Gelsenkirchen (DE); Ivo Robert Kühnrich, Dortmund (DE); Martina Ludwig, Recklinghausen (DE); Dirk Mackowiak, Borken (DE); Jörg Zöllner, Recklinghausen (DE); Stephan Kohlstruk, Gladbeck (DE)

(73) Assignee: Evonik Operations GmbH, Essen (DE)

(21) Appl. No.: 19/573,017

(22) Filed: Mar. 20, 2026

(30) Foreign Application Priority Data
Mar. 21, 2025 (EP) 25165266.5

(51) Int. Cl. C07C 45/74

(52) U.S. Cl. CPC C07C 45/74; C07C 2601/16

(57) ABSTRACT

The present invention provides a liquid phase process for preparing isophorone, in which acetone is continuously reacted under catalysis with addition of mesityl oxide, wherein at least 50% by weight of the mesityl oxide added is added after at least 50% of the reactor volume has been passed through.

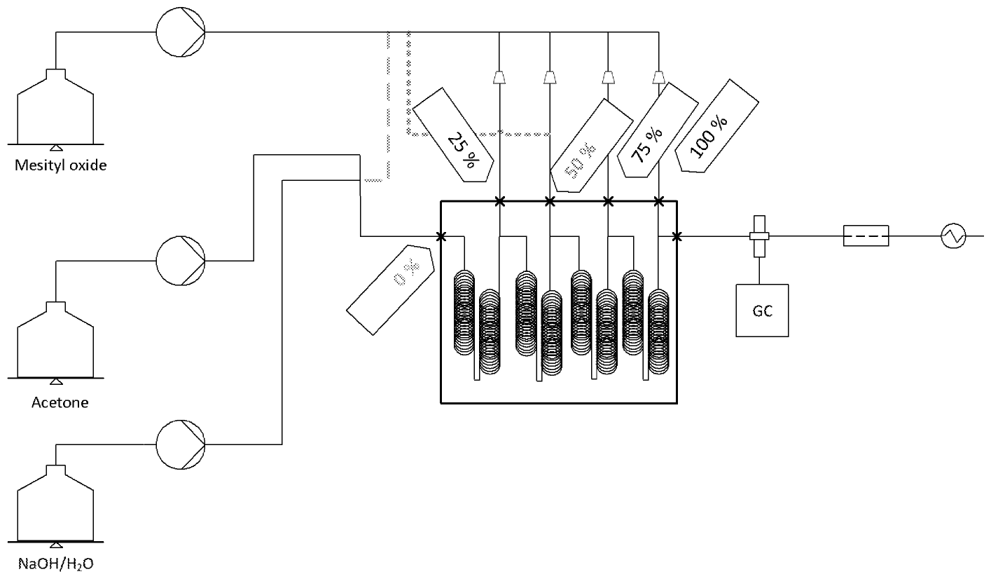

The present invention relates to a liquid phase process for preparing isophorone.

Isophorone is an important industrial chemical that is of relevance because, inter alia, it serves as a reactant for the synthesis of isophorone nitrile, which in turn is used for the synthesis of isophorone diamine, which is used as an epoxy hardener, and of the isophorone diisocyanate preparable in turn therefrom. The last of the aforementioned compounds is of great relevance for polyurethane chemistry. Processes for preparing isophorone are therefore of great interest.

JP H09-157207 A and JP H09-157208 A disclose processes for preparing isophorone from acetone in the gas phase. The reaction is performed in the presence of mesityl oxide. A disadvantage of the stated method is that high temperatures are required to carry out the reaction in the gas phase. Another disadvantage is that the heterogeneous catalysts (zeolites) used have to be replaced and regenerated at regular intervals.

These disadvantages can be overcome by synthesizing isophorone from acetone under base catalysis in a liquid phase process: DE 1 205 525 B discloses a process for preparing isophorone in the liquid phase, in which acetone is condensed to form isophorone. Mesityl oxide, which is obtained in considerable amounts, and overcondensates can be removed by treating the by-products with an aqueous alkali solution at relatively high temperatures. It is disclosed that mesityl oxide is completely degraded at these temperatures.

DE 26 45 281 A1 discloses a liquid phase process for preparing isophorone, in which acetone, water and an alkali metal catalyst are reacted. The isophorone is removed by distillation.

U.S. Pat. No. 5,849,957 A discloses a process for preparing isophorone from acetone in the presence of a magnesium oxide catalyst. The resultant by-products include mesityl oxide and can be recycled.

U.S. Pat. No. 2,419,051 A discloses a process for providing isophorone and acetone, in which autocondensation products of acetone having a higher boiling point than isophorone are reacted with an alkali metal hydroxide to produce isophorone and acetone.

GB 733,650 A discloses that acetone can be reacted under base catalysis to form isophorone and the intermediate mesityl oxide and also higher condensation products. In a preferred embodiment, the higher condensation products are recycled into the reaction mixture.

CN 103435461 A discloses a process for preparing isophorone from acetone, in which unreacted acetone can be recycled into the reaction.

DE 25 20 681A1 , too, discloses a process for preparing isophorone, in which acetone is reacted in the presence of potassium hydroxide to form isophorone. The resultant by-products can be converted back to acetone by hydrolysis and are returned to the reaction.

CN 102367223 A discloses a process for preparing isophorone, in which acetone is condensed in the presence of a catalyst. Unreacted acetone is removed and recycled into the reaction. High boilers having 12 or more carbon atoms undergo hydrolysis.

U.S. Pat. No. 2,344,226 A discloses a process for preparing isophorone, in which acetone is heated in the presence of an alkali metal hydroxide. The mesityl oxide that is formed as an intermediate, together with components such as diacetone alcohol, phorone and higher condensation products that are likewise formed, can be re-added to the reaction mixture.

U.S. Pat. No. 4,086,188 A discloses processes for preparing mesityl oxide and isophorone. It is disclosed that it is desirable for the ratio of mesityl oxide formed to isophorone formed to be less than 1, since it is otherwise necessary to recycle excess mesityl oxide.

DE 10 2010 062 587 A1 discloses a liquid phase process for preparing isophorone from acetone, in which an aqueous alkali metal hydroxide solution can be used as a catalyst. The resultant light boilers comprising mesityl oxide and diacetone alcohol can be recycled into the reactor together with unreacted acetone.

WO 2012/076317A1 , too, discloses a liquid phase process for preparing isophorone from acetone, in which an aqueous alkali metal hydroxide solution can be used as a catalyst. Here too, light boilers comprising mesityl oxide and diacetone alcohol that are obtained can be recycled into the reaction.

It was found that the amounts of acetone required in the aldol condensation of acetone to form isophorone could be reduced by adding mesityl oxide, in particular by recycling mesityl oxide formed during the reaction. However, in the processes previously known in the prior art, the conversion based on acetone, mesityl oxide and diacetone alcohol was reduced, which is disadvantageous.

It is therefore an object of the present invention to provide a liquid phase process for preparing isophorone from acetone with supply of mesityl oxide, by means of which higher conversions can be achieved. It has been found that, surprisingly, the present object can be achieved when at least 50% by weight of the mesityl oxide added is added after at least 50% of the reactor volume has been passed through.

The present invention therefore provides a liquid phase process for preparing isophorone, in which acetone is continuously reacted in the presence of a catalyst with addition of mesityl oxide, in which at least 50% by weight of the mesityl oxide added is added after at least 50% of the reactor volume has been passed through.

The process according to the invention is a liquid phase process. Relevant processes involve performing the reaction in the liquid phase. The process according to the invention is therefore performed neither in the gas phase nor in supercritical acetone.

Preferably, the reaction is performed at temperatures of 100-250° C., preferably 120-250° C. and particularly preferably 180-250° C., and pressures of 5-50 bar, preferably 10-50 bar and particularly preferably from 20-50 bar, and any combination of the values specified is possible.

The reaction is effected in the presence of a catalyst. The catalysts used may be homogeneous or heterogeneous catalysts. Preference is given to using a homogeneous catalyst.

The condensation of acetone to form isophorone is preferably effected under base catalysis. Preferably, the base used is an alkali metal hydroxide solution. Yet more preferably, the basic catalyst is selected from the group consisting of NaOH and KOH.

Preferably, the content of base, in particular alkali metal hydroxide solution, more preferably NaOH or KOH, is less than or equal to 5% by weight, preferably less than or equal to 1% by weight and particularly preferably less than or equal to 0.5% by weight, based on the total mass of the reaction composition. Most preferably, the catalyst used is NaOH in an amount of 0.005% to 0.5% by weight.

Especially when the catalyst used is an aqueous alkali metal hydroxide solution, the reaction will take place in the presence of water. In addition, water will be formed during the reaction owing to the process of condensation. Preferably, the water content of the reaction mixture, defined by the amount of water supplied to the reactor, based on the amounts of acetone, water and mesityl oxide supplied, is less than or equal to 40% by weight and preferably less than or equal to 30% by weight.

Preferably, the acetone content in the mixture, defined by the amount of acetone supplied to the reactor, based on the amounts of acetone, water and mesityl oxide supplied, is greater than or equal to 25% by weight, more preferably greater than or equal to 50% by weight and yet more preferably greater than or equal to 65% by weight.

The aldol condensation of acetone to form isophorone is effected with addition of mesityl oxide. Preferably, the mesityl oxide content, based on the amounts of acetone, water and mesityl oxide supplied, is 0.1-50% by weight, more preferably 0.5-25% by weight and yet more preferably 0.8-10% by weight.

The reaction is performed continuously in a reactor. A continuous reaction regimen is to be understood herein to mean a continuous supply of reactant and catalyst into the reactor while continuously discharging product and unreacted reactants and intermediates.

Reactors preferably usable for the continuous reaction regimen can be selected from the group consisting of tubular reactors, stirred tank cascades, fixed bed reactors, trickle bed reactors, slurry reactors, pressure distillation reactors, reactive distillation reactors, microstructured reactors, membrane reactors, chamber reactors, static mixers and bubble columns.

Preferably, the reactor is a flow reactor.

According to the invention, at least 50% by weight of the mesityl oxide added is added after at least 50% of the reactor volume has been passed through. The reactor volume is to be understood here to mean the volume within which acetone is reacted in the presence of the catalyst and with itself and with any intermediates present (including mesityl oxide) to form isophorone. More preferably, the reactor volume is the volume of the reactor within which acetone is contacted in the presence of catalyst with itself and with any intermediates present (including mesityl oxide) at a pressure and a temperature selected from the preferred pressure and temperature ranges already mentioned above. Especially when using a flow reactor or tubular reactor having a cross-section or tube of constant diameter, the position at which x% of the reactor volume has been passed through corresponds to x% of the length of the reactor.

The addition of the mesityl oxide may be effected continuously, though it need not be. It is also conceivable to achieve positive properties by effecting the addition of the mesityl oxide semi-continuously or discontinuously. However, preference is given to adding the mesityl oxide continuously as well.

Particularly high conversions can be achieved when at least 60% by weight, more preferably at least 70% by weight and yet more preferably at least 75% by weight of the mesityl oxide added is added after at least 50% of the reactor volume has been passed through.

The addition made in accordance with the invention of at least 50% by weight, preferably at least 60% by weight, more preferably at least 70% by weight and yet more preferably at least 75% by weight of the mesityl oxide after at least 50% of the reactor volume has been passed through may be made via one metering point or two or more metering points, all of which are located at a position after at least 50% of the reactor volume has been passed through. Preferably, the addition of at least 50% by weight, more preferably at least 60% by weight, yet more preferably at least 70% by weight and most preferably at least 75% by weight of the mesityl oxide after at least 50% of the reactor volume has been passed through is made via 2 or more, more preferably 3 or more and most preferably 4 or more metering points, all of which are located at a position after at least 50% of the reactor volume has been passed through. A preferred upper limit for the number of said metering points is preferably 20, more preferably 15 and yet more preferably 10.

The remaining percentages of the mesityl oxide added that make up 100% by weight (i.e. less than or equal to 50% by weight in the case of at least 50% by weight of mesityl oxide added after 50% of the reactor volume, i.e. less than or equal to 40% by weight in the case of at least 60% by weight of mesityl oxide added after 50% of the reactor volume, i.e. less than or equal to 30% by weight in the case of at least 70% by weight of mesityl oxide added after 50% of the reactor volume, i.e. less than or equal to 25% by weight in the case of at least 75% by weight of mesityl oxide added after 50% of the reactor volume) are added before at least 50% of the reactor volume has been passed through. Here too, the addition may be made via one or more, preferably 2 or more and more preferably 3 or more metering points. A preferred upper limit for the number of said metering points is preferably 15, more preferably 10 and yet more preferably 5.

Preferably, mesityl oxide is not added before at least 25% of the reactor volume has been passed through, since this achieves particularly good conversions.

Preferably, mesityl oxide is not added before at least 50% of the reactor volume has been passed through, since this achieves particularly good conversions combined with particularly good selectivities.

Preferably, the mesityl oxide supplied comes from working up the reaction mixture containing materials of value from the liquid phase process for preparing isophorone.

The reaction mixture containing materials of value after the total reactor volume has been passed through can be worked up therefor and separated into the individual components. Besides isophorone, what are obtained are acetone, the light boilers (i.e. substances having a lower boiling point than isophorone, in particular diacetone alcohol and mesityl oxide), higher condensation products (overcondensates) of acetone (i.e. having a higher boiling point than isophorone, in particular xylitones and isoxylitones), water and catalyst.

In principle, the separation into the individual components can be performed using all separation methods, in particular distillation, flash evaporation, crystallization, extraction, sorption, permeation, phase separation or combinations thereof, continuously or batchwise, in one or more stages. Preferably, the separation is effected by distillation in one or more apparatuses.

More preferably, the removal of the individual fractions is effected by a reactive distillation downstream of the reactor, preferably in a reactive distillation column.

Particularly preferably, the removal is performed in a separate space from the isophorone synthesis (reaction) in a reactive distillation column with a sidestream withdrawal.

Preferably, the removal is performed so as to obtain the following three fractions:
i) a fraction of unreacted acetone, water and light boilers (in particular diacetone alcohol and mesityl oxide)
ii) a fraction in which colouring substances in particular are enriched, and
iii) a fraction comprising in particular isophorone, more highly condensed products, water and catalyst, referred to as stream of value. This fraction is then subjected to hydrolysis.

Fraction i) was previously preferably condensed before being recycled into the reactor for reaction. In a further preferred embodiment, it was withdrawn as a vapour stream comprising essentially acetone, water and low boilers, essentially diacetone alcohol and mesityl oxide, condensed and re-added to the reactor with the acetone, water and optionally catalyst feedstocks.

In a preferred embodiment of the present invention, the addition of the mesityl oxide is effected by adding a fraction comprising acetone, water and light boilers that is obtainable by distillation of the reaction mixture containing materials of value after the total reactor volume has been passed through. In principle, the mesityl oxide can therefore be added together with other light boilers, in particular diacetone alcohol, and also water and acetone. Any light boilers present other than mesityl oxide are not taken into account when determining the percentage content of mesityl oxide, i.e. the specified percentage value is always based on the amounts of acetone, water and mesityl oxide supplied. More preferably, this fraction therefore corresponds to the above-mentioned fraction i).

Preferably, the fraction comprises proportions of 60-85% by weight of acetone, 10-25% by weight of water, 0.5-10% by weight of diacetone alcohol and 0.5-10% by weight of mesityl oxide.

More preferably,
proceeding from the fraction comprising acetone, water and light boilers, preferably fraction i),
   -acetone and water are
      removed from the light boilers, preferably by distillation, and
      added to the start of the reactor.

In this likewise preferred embodiment, the addition of the mesityl oxide is therefore effected by adding the light boilers, preferably consisting essentially of a mixture of mesityl oxide and diacetone alcohol, that are preparable by
distillation of the reaction mixture containing materials of value after the total reactor volume of the reactor has been passed through to obtain a fraction comprising acetone, water and light boilers, and
subsequent removal, preferably distillative removal, of acetone and water from said fraction.

In a preferred embodiment, fraction ii) is withdrawn as a sidestream of the distillation column, preferably of a reactive distillation column, optionally neutralized and worked up further. This fraction can be purified further and the materials of value present can be recycled into the process. For the workup, it is possible to use all standard separation methods, for example distillation, flash evaporation, crystallization, extraction, sorption, permeation, phase separation or combinations thereof. The purification can be performed continuously or batchwise, in one or more stages. Preferably, the purification is achieved by distillation. Particularly preferably, the purification is achieved by a combination of neutralization or extraction and subsequent distillation, preferably in a reactive distillation column. Preferably, the worked-up phase is conducted into the hydrolysis with the products of value composed of isophorone and high boilers with or without catalyst. Any further phase composed of products of value, essentially comprising acetone, diacetone alcohol and mesityl oxide, that is obtained is preferably recycled into the reaction. However, this is preferably not done because of the relatively high apparatus requirements. Any residues obtained can be used for heat recovery.

Fraction iii) is preferably obtained in the bottoms of the distillation column, where it can be withdrawn to obtain the stream of value.

The stream of value of the aldol condensation is then preferably b) hydrolysed, i.e. subjected to hydrolysis. The aim of the hydrolysis is partial or complete conversion of by-products present in the stream of value into isophorone, acetone or other usable products of value. The hydrolysis can be performed in all standard reactors, distillation columns or combinations thereof. Preferably, the hydrolysis is performed as a reactive distillation, in which the low boilers formed, essentially consisting of diacetone alcohol and mesityl oxide, are removed directly from the hydrolysis zone and recycled into the reaction, and are thus no longer available for side reactions in the hydrolysis.

Most preferably, the hydrolysis of fraction iii) can be effected in an apparatus by a reactive distillation, preferably in a reactive distillation column, with simultaneous separation of the reaction mixture into fractions i) to iii), such that the products formed are correspondingly separated at the same time and fraction iii) is hydrolysed.

The hydrolysis can be performed in all mixing ratios of the organic components with water, with or without catalyst. The water concentration in the hydrolysis is 30.1-99.9% by weight, based on the total mass of the constituents present. In the case of homogeneous catalysis, the catalyst used in the hydrolysis is preferably that which is also used in the aldol condensation. Preference is given to choosing catalyst concentrations of 0.001-10% by weight, particularly preferably of 0.05-1% by weight, based on the total mass of the composition. The pressure in the hydrolysis reactor is 1-200 bar, preferably 20-60 bar. Particularly preferably, the hydrolysis is performed at least at the same pressure as that in the isophorone synthesis step (reaction). The hydrolysis temperature is 100-300° C., preferably 210-260° C. More preferably in the case of use of a reactive distillation column, a temperature or temperature profile will be established according to the boiling temperatures in the bottoms and at the individual separation or reaction stages.

The hydrolysis can be performed in one or more apparatuses, in one or more stages.

After it has more preferably been discharged from the hydrolysis reactor of the reactive distillation column and cooled, the hydrolysate obtained is then preferably subjected to a phase separation c).

The phase separation can yield an essentially organic fraction i) and an essentially aqueous fraction ii). In the case of homogeneous catalysis of the aldol condensation, the aqueous fraction ii) also contains the catalyst. Customary phase-separation vessels with or without internals can be used for the phase separation. The phase separation is effected at a temperature between 0-200° C., preferably at 0-100° C. and particularly preferably at 20-70° C., and at a pressure of 1-150 bar, preferably 20-60 bar and particularly preferably at the same pressure as that in the hydrolysis.

The essentially organic phase i) comprising the isophorone target product can be optionally neutralized and purified by customary methods, so as to obtain an isophorone with the desired purity and colour stability. It is possible here to use all standard separation methods, in particular distillation, flash evaporation, crystallization, extraction, sorption, permeation, phase separation and combinations thereof. The purification can be performed continuously or batchwise, in one or more stages, under pressure or under reduced pressure. Preferably, the purification is achieved by distillation. Particularly preferably, the purification is achieved by a combination of neutralization or extraction and subsequent distillation.

The essentially aqueous fraction ii) can undergo wastewater cleaning. Here, the water of reaction as the main constituent is separated, with or without the catalyst, from any organic components still dissolved, for example isophorone, acetone and more highly condensed products. The wastewater cleaning operation is preferably performed in one or more distillation columns.

The isophorone preparable by the process according to the invention is suitable for a multitude of applications. In particular, it is suitable for the preparation of isophorone nitrile. Isophorone nitrile can furthermore be advantageously used for the synthesis of isophorone diamine, especially by means of aminating hydrogenation. Isophorone diamine can furthermore be converted into isophorone diisocyanate.

The present invention therefore provides not just the isophorone obtainable by the process according to the invention, but also the use thereof for preparing isophorone nitrile, isophorone diamine and/or isophorone diisocyanate.

Unless stated otherwise, all stated percentages (%) are percentages by mass.

The examples recited below describe the present invention by way of example without any intention to limit the invention, the scope of application of which is apparent from the entirety of the description and the claims, to the embodiments specified in the examples.

### BRIEF DESCRIPTION OF THE DRAWINGS

The FIGURE shows an example of a continuously operated laboratory installation in accordance with this disclosure.

### EXAMPLES

The experiments were performed in a continuously operated laboratory installation (see the Figure). The installation consisted of reservoirs for acetone, aqueous sodium hydroxide solution and mesityl oxide, pumps for metering the reactants, a thermal oil-heated tubular reactor, a pressure regulator, and also online GC and a sampling point for offline analyses. The feeds were metered in by HPLC pumps within a range of volumetric flow rates of 0.4-5.4 mL/min. The tubular reactor had a volume of 235 mL and was composed of 16 separate spiral units with a total number of 256 windings. Efficient mixing was achieved by inverting the flow direction after every 6.25% of the reactor volume. Mesityl oxide was metered into the reactor via T-pieces after 0%, 25%, 50%, 75% and 100% of the reactor volume. For precise control of volumetric flow rate, the addition was made via suitable restrictors. Reaction progress was monitored analytically by online gas chromatography using a pressure-stable piston injector. The online measurements were verified by taking sample material after a reaction had completed and testing it by offline gas chromatography.

### 1. Preparation of Isophorone by Reaction of Acetone Under Base Catalysis with Addition of Mesityl Oxide at 0% of the Reactor Volume

Acetone (5.39 mL/min) and aqueous sodium hydroxide solution (0.169% by weight in water, 1.42 mL/min) were combined upstream of the reactor. Mesityl oxide (0.42 mL/min, 6% by weight of the total feed) was fully metered in at 0%. The reaction was effected in a tubular reactor at 200° C. and 35 bar and a resulting average residence time of 35 minutes. The conversion based on acetone, mesityl oxide and diacetone alcohol was 8.0% and the selectivity for isophorone was 89.6%.

### 2. Preparation of Isophorone by Reaction of Acetone Under Base Catalysis with Addition of Mesityl Oxide at 50% of the Reactor Volume

Acetone (5.39 mL/min) and aqueous sodium hydroxide solution (0.169% by weight in water, 1.42 mL/min) were combined upstream of the reactor. Mesityl oxide (0.42 mL/min, 6% by weight of the total feed) was fully metered in after 50% of the reactor volume. The reaction was effected in a tubular reactor at 200° C. and 35 bar and a resulting average residence time of 35 minutes. The conversion based on acetone, mesityl oxide and diacetone alcohol was 8.4% and the selectivity for isophorone was 89.5%.

### 3. Preparation of Isophorone by Reaction of Acetone Under Base Catalysis with Addition of Mesityl Oxide at 25%, 50%, 75% and 100% of the Reactor Volume

Acetone (5.39 mL/min) and aqueous sodium hydroxide solution (0.169% by weight in water, 1.42 mL/min) were combined upstream of the reactor. Mesityl oxide (overall: 0.42 mL/min, 6% by weight of the total feed) was metered in stepwise after 25%, 50%, 75% and 100% (0.11 mL/min in each case, 1.5% by weight of the total feed) of the reactor volume. The reaction was effected in a tubular reactor at 200° C. and 35 bar and a resulting average residence time of 35 minutes. The conversion based on acetone, mesityl oxide and diacetone alcohol was 8.6% and the selectivity for isophorone was 86.4%.

1. Liquid phase process for preparing isophorone, in which acetone is continuously reacted in the presence of a catalyst with addition of mesityl oxide,
wherein at least 50% by weight of the mesityl oxide added is added after at least 50% of the reactor volume has been passed through.

2. Process according to claim 1,
wherein the reaction is effected at temperatures of 100-250° C. and a pressure of 5-50 bar.

3. Process according to claim 1,
wherein the catalyst is selected from NaOH and KOH.

4. Process according to claim 1,
wherein the reactor is selected from the group of reactors consisting of tubular reactors, stirred tank cascades, fixed bed reactors, trickle bed reactors, slurry reactors, pressure distillation reactors, reactive distillation reactors, microstructured reactors, membrane reactors, chamber reactors, static mixers and bubble columns.

5. Process according to claim 1,
wherein the reactor is a flow reactor.

6. Process according to claim 1,
wherein at least 60% by weight of the mesityl oxide added is added after at least 50% of the reactor volume has been passed through.

7. Process according to claim 1,
wherein the addition of the at least 50% by weight of the mesityl oxide after at least 50% of the reactor volume has been passed through is made via two or more metering points, all of which are located at a position after at least 50% of the reactor volume has been passed through.

8. Process according to claim 1,
wherein mesityl oxide is not added before at least 25% of the reactor volume has been passed through.

9. Process according to claim 8,
wherein mesityl oxide is not added before at least 50% of the reactor volume has been passed through.

10. Process according to claim 1,
wherein the mesityl oxide supplied comes from working up the reaction mixture containing materials of value from the liquid phase process.

11. Process according to claim 10,
wherein the addition of the mesityl oxide
is effected by adding a fraction comprising acetone, water and light boilers that is obtainable by distillation of the reaction mixture containing materials of value after the total reactor volume has been passed through.

12. Process according to claim 10,
wherein the addition of the mesityl oxide
is effected by adding the light boilers that are preparable by
distillation of the reaction mixture containing materials of value after the total reactor volume has been passed through to obtain a fraction comprising acetone, water and light boilers, and
subsequent removal of acetone and water from said fraction.
